(19) United States
(12) Plant Patent Application Publication
(10) Pub. No.: US 2004/0158895 P1
(43) Pub. Date: Aug. 12, 2004

(54) Miniature rose plant 'POULsabel'

(76) Inventors: L. Pernille Olesen, Fredensborg (DK); Mogens N. Olesen, Fredensborg (DK)

Correspondence Address: Poulsen Roser Pacific, Inc., 620 S. Front Street, Centre Point, OR, 97502, US

(21) Appl. No.: 10/683,876

(22) Filed: Apr. 1, 2004

Related U.S. Application Data
(63) Continuation of application No. 10/136,451, filed on Apr. 29, 2002, now abandoned.
Application No. 10/136,451 is a continuation of application No. 09/607,333, filed on Jun. 30, 2000, now abandoned.

(51) Int. Cl. A01H 5/00

(52) U.S. Cl. PLT/122

(57) ABSTRACT

A new miniature rose plant which has abundant, non-fading, red-purple flowers and attractive foliage. The variety successfully propagates from softwood cuttings and is suitable for year round production in commercial glasshouses. This new and distinct variety has shown to be uniform and stable in the resulting generations from asexual propagation.

### SUMMARY OF THE INVENTION

### Botanical Classification

Rosa hybrida

### Variety Denomination

‘POULsabel’

The present invention constitutes a new and distinct variety of miniature rose plant which originated from a controlled crossing between an unnamed seedling and ‘POULvic’ (U.S. Pat. No. 8,012, issued on Oct. 27, 1994). The two parents were crossed and the resulting seeds were planted in a controlled environment. The new variety is named ‘POULsabel’.

The new rose may be distinguished from its seed parent, an unnamed seedling, by the following combination of characteristics:

1. The petals of the unnamed seedling are yellow, while those of ‘POULsabel’ are red-purple.

2. The unnamed seedling is a floribunda rose, where ‘POULsabel’ is a miniature rose.

The new variety may be distinguished from its pollen parent, ‘POULvic’, created by the same inventors, by the following combination of characteristics:

1. ‘POULvic’ has smaller flowers than ‘POULsabel’.

2. The foliage of ‘POULvic’ is smaller than that of ‘POULsabel’.

3. The amount of flowers ‘POULvic’ produces is greater than that which ‘POULsabel’ produces.

The objective of the hybridization of this rose variety for commercial greenhouse culture was to create a new and distinct variety with unique qualities, such as:

1. Uniform and abundant flowers;

2. Vigorous and compact growth;

3. Year-round flowering under glasshouse conditions;

4. Suitability for production from softwood cuttings in pots;

5. Durable flowers and foliage which make a variety suitable for distribution in the floral industry.

The combination of qualities of this variety represents significant improvement over previously available commercial cultivars of this type and distinguishes ‘POULsabel’ from all other varieties of which we are aware.

As part of their rose development program, L. Pernille Olesen and Mogens N. Olesen germinated the seeds from the aforementioned hybridization and conducted evaluations on the resulting seedlings in a controlled environment in Fredensborg, Denmark.

‘POULsabel’ was selected by the inventors as a single plant from the progeny of the hybridization in Fredensborg, Denmark.

Asexual reproduction of ‘POULsabel’ by cuttings and traditional budding was first done by L. Pernille and Mogens N. Olesen in Fredensborg, Denmark. This initial and other subsequent propagations conducted in controlled environments have demonstrated that the characteristics of ‘POULsabel’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, stems, and a plant of ‘POULsabel’.

Specifically illustrated in SHEET 1:

1. Stem or entire plant showing branching and the attachment of leaves, buds, and peduncles;

2. Flower bud, partially opened bud, and open bloom;

3. Flower petals, detached;

Specifically illustrated ion SHEET 2:

4. Sepals, receptacle, and pedicel;

5. Stem with foliage as well as a bare stem exhibiting thorns;

6. Leaves.

### DETAILED DESCRIPTION OF THE VARIETY

The following is a description of ‘POULsabel’, as observed in its growth in glasshouses in Fredensborg, Denmark; Burlington, Canada; Half Moon Bay, Calif., and in a field nursery in Jackson County, Oregon, on plants aged 16 weeks. Color references are made using The Royal Horticultural Society (London, England) Colour Chart, 1995, except where common terms of color are used.

For a comparison, several physical characteristics of the rose variety ‘POULypso’, a miniature rose variety from the same inventors described and illustrated in U.S. Plant patent application Ser. No. 09/138,034 dated Aug. 18, 1998 are compared to ‘POULsabel’ in Chart 1.

CHART 1

| | ‘POULsabel’ | ‘POULypso’ |
|---|---|---|
| Bud color at ¼ | Red-Purple Group 61B | Red Group 45A |
| open. | | |
| Upper Petal | Red-Purple Group 57A | Red Group 44B |
| Surface, open | | |
| bloom. | | |
| Reverse petal | Red-Purple Group 61B | Red Group 53C |
| surface, open | | |
| bloom. | | |

Parents: Unnamed seedling×‘POULvic’.

Classification:

Botanical.—Rosa hybrida.

Commercial.—Miniature.

### FLOWER AND FLOWER BUD

Blooming habit: Recurrent.

Flower bud:

Size.—Upon opening, 25 to 30 mm in length from base of receptacle to end of bud.

Bud form.—Long; broad-based.

Bud color.—As sepals unfold, Red Group53A, outer petals Red-Purple Group 60B. Red-Purple Group 61B at ¼ opening.

Sepals.—Sepal length is 12 to 15 mm. Sepal width is 7 to 10 mm. Green Group 143A and C. Moderate foliaceous appendages on three of the five sepals. Surfaces of sepals moderately pubescent. Stipitate glands are present on the margins of two of the five sepals with fine white hairs present on all five of the sepal margins.

Receptacle.—Surface: Smooth. Shape: Urn-shaped. Size: Small; 6 mm (h)×12 mm (w). Color: Yellow-Green Group 144 A and B.

Peduncle.—Surface: Generally with some stipitate glands present. Length: 48 mm average length. Color: Yellow-Green Group 144A and B. Strength: Upright. Diameter: 4 mm.

Borne.—Singly.

Flower bloom:

Fragrance.—Lightly floral.

Duration.—As a pot plant, flowers last from 15 to 21 days. As a cut flower 6 to 7 days.

Size.—Medium for a 12.5 cm pot rose. Average flower diameter is 55 mm when open.

Form.—

Shape of flower when viewed from the side.—Upon opening, upper part: Flat. Upon opening, lower part: Flat. Open flower, upper part: Flat. Open flower, lower part: Flat.

Petalage.—Average range: 30 to 35 petals under normal conditions with 2 to 4 petaloids.

Color:

Upon opening, petals.—Outermost petals: Upper Surface: Green-White Group 157A at point of attachment; Green-white Group 155C in basal zone. Reverse Side: Green-White Group 157A. Some petals show striping of White Group 155C in center of petal. Innermost petals: Upper Surface: White Group 155C and D. Reverse Side: White Group 155C and D.

After opening petals.—Petals: Upper surface: Red-Purple Group 57A. Reverse Side: Red-Purple Group 61B.

After opening basal petal spots.—Outermost petals: Outer Side: Green-White Group 157A at point of attachment; Green-white Group 155C in basal zone. Inner Side: Green-White Group 157A. Some petals show striping of White Group 155C in center of petal. Innermost petals: Outer Side: White Group 155C. Inner Side: White Group 155C.

General tonality: On open flower Red Group 53A. No change in the general tonality at the end of the 5^{th} day. Afterwards, general tonality is Red Group 53B.

Petals:

Petal reflex.—Petals reflex somewhat.

Petal margin.—Entire.

Shape.—Deltoid.

Petaloids.—3 to 4.

Thickness.—Thick; soft with a velvet sheen.

Arrangement.—Not imbricated.

Size.—15 mm (1)×13 mm (w).

Reproductive organs:

Pollen.—Color: Yellow-Orange Group 21A. Quantity: Average.

Anthers.—Size: 2 mm. Color: Greyed-Brown Group 199D. Quantity: 25 to 35.

Filaments.—Color: Yellow Group 2C. Length: 3 to 5 mm.

Stigmas.—Very slightly superior in location to anthers. Color: Yellow-Green Group 1D.

Styles.—Color: Yellow-Green Group 1D. Length: 5 to 6 mm.

Hips.—None observed.

### PLANT

Plant growth: Vigorous, compact, and upright. When grown as a 12 cm pot plant, the average height of the plant itself is 26 cm and the average width is 23 cm. When grown as a budded field grown plant on rosa multiflora understock, the average height of the plant itself is 39 cm and the average width is 34 cm.

Stems:

Color.—Young wood: Green Group 137A and B. Older wood: Green Group 137B.

Thorns.—Incidence: Few. Size: Average length: 5 to 7 mm. Color: Greyed-Purple Group 185B. Shape: Linear.

Surface.—Young wood: Smooth. Older wood: Smooth.

Length.—Under typical greenhouse production conditions, stem length is 25 to 30 cm. Internode length varies from 8 to 10 cm.

Plant foliage: Normal number of leaflets on normal leaves in middle of the stem: 5 leaflets.

Leaf size.—110 mm (1)×82 mm (w).

Quantity.—Average.

Color.—Upper Leaf Surface: Green Group 139A. Lower Leaf Surface: Green Group 139C. Juvenile foliage: Upper surface is Yellow-Green Group 144C. Lower surface is Green Group 139C. Anthocyanin intonation: Location: Leaf margins of juvenile foliage, petiole, margin. Color: Greyed-Red Group 181A.

Plant leaves and leaflets:

Stipules.—Size: 10 to 13 mm. Color: Yellow-Green Group 146A. Stipitate glands: Present on margins.

Petiole.—Length: 17 mm. Color: Yellow-Green Group 146A. Underneath: Yellow-Green Group 146A. No prickles observed. Margins: Margins are smooth with very few stipitate glands.

Rachis.—Color: Yellow-Green Group 146A. Underneath: Yellow-Green Group 146A. No prickles observed. Margins: Stipitate glands present, every 2 to 3 mm.

Leaflet.—Edge: Serrated. Shape: Broadly ovate. Other: On plants grown under high light conditions, juvenile leaflet margins are Greyed-Red Group 181A.

Disease resistance: Above average resistance to mildew, rust and black spot under normal growing conditions in Jackson County, Oregon.

Cold hardiness: ‘POULsabel’ has been found to be resistant to damage from cold, heat and drought damage in USDA Zone 7.

1. A new and distinct variety of rose plant of the miniature class, substantially as herein illustrated and described as a distinct and novel rose variety due to its abundant, dark red (RHS Red Group 53A) flowers, vigorous and compact growth, year round flowering under glasshouse conditions, resistance to disease, winter and heat hardiness, suitability for production from softwood cuttings in pots as well as budding as a field grown plant, suitability for production as a tree rose, light floral scent, and durable flowers and glossy, green (RHD Green Group 137A and Yellow-Green Group 139A) foliage which make the variety suitable for distribution in the floral industry.
